(19) United States
(12) Patent Application Publication
(10) Pub. No.: US 2026/0130866 A1
(43) Pub. Date: May 14, 2026

(54) IMMEDIATE RELEASE AMLODIPINE UBIQUINONE COMPOSITION FOR ENHANCED IMMUNOMODULATORY ACTIVITY

(71) Applicants: ABIDA KHAN, Arar (SA); BANDAR THEYAB ALENEZI, Rafha (SA); MUHAMMAD KHAN SARFRAZ, Al Ain (AE); MOSAB ARAFAT, Al Ain (AE); ZIA UR REHMAN, Jazan (SA); MUHAMMAD IRFAN SIDDIQUE, Rafha (SA); FAIYAZ SHAKEEL, Riyadh (SA); Syed Mohammed Basheeruddin ASDAQ, Riyadh (SA); SEEMA GARG, NOIDA (IN); MOHD IMRAN, Arar (SA)

(72) Inventors: ABIDA KHAN, Arar (SA); BANDAR THEYAB ALENEZI, Rafha (SA); MUHAMMAD KHAN SARFRAZ, Al Ain (AE); MOSAB ARAFAT, Al Ain (AE); ZIA UR REHMAN, Jazan (SA); MUHAMMAD IRFAN SIDDIQUE, Rafha (SA); FAIYAZ SHAKEEL, Riyadh (SA); Syed Mohammed Basheeruddin ASDAQ, Riyadh (SA); SEEMA GARG, NOIDA (IN); MOHD IMRAN, Arar (SA)

(21) Appl. No.: 19/438,692

(22) Filed: Jan. 2, 2026

(51) Int. Cl. A61K 31/122; A61K 9/20; A61K 31/4422

(52) U.S. Cl. CPC A61K 31/122; A61K 9/2009; A61K 9/2013; A61K 9/2027; A61K 9/2054; A61K 9/2095; A61K 31/4422

(57) ABSTRACT

The present invention relates to an immediate-release oral pharmaceutical composition comprising Amlodipine and Ubiquinone (coenzyme Q10) for enhanced immunomodulatory activity. The formulation includes Amlodipine besylate (5 mg), Ubiquinone (coenzyme Q10) (50 mg), and pharmaceutically acceptable excipients to form a 500 mg tablet capable of rapid disintegration and drug release. The composition demonstrates superior cell-mediated and innate immune enhancement compared to standard immunostimulants, as evidenced by delayed-type hypersensitivity and carbon clearance assays. The synergistic combination not only restores immune competence in post-viral immune suppression syndrome (PVISS) and immune fatigue but also improves macrophage activation and lymphocyte response. The formulation is stable, effective, and suitable for once-daily oral administration.

### TECHNICAL FIELD

The present invention relates to the field of pharmaceutical and nutraceutical formulations. More particularly, it pertains to a novel composition comprising Amlodipine besylate and ubiquinone (coenzyme Q10) designed to provide immunomodulatory and antioxidant effects, specifically targeting conditions such as post-viral immune suppression syndrome (PVISS), chronic immune fatigue, and oxidative stress-related immune dysfunction.

### BACKGROUND OF THE INVENTION

Post-viral immune suppression syndrome (PVISS) is a clinically observed consequence of viral infections such as influenza, Epstein-Barr virus, and most recently, COVID-19. It is characterized by a significant reduction in lymphocyte proliferation, impaired macrophage function, and elevated oxidative stress. Current therapeutic approaches predominantly offer symptomatic relief and do not effectively restore immune homeostasis or cellular immune function.

Amlodipine besylate, a third-generation calcium channel blocker commonly used to manage hypertension and angina, has been recently reported to exhibit anti-inflammatory and immune-modulating properties, including effects on T-cell activation and endothelial immune signaling. Ubiquinone (coenzyme Q10), although classified as a bioactive nutraceutical rather than a pharmaceutical ingredient, plays a key role in mitochondrial function, cellular energy metabolism, and acts as a potent antioxidant that supports immune cell viability and function.

Despite independent evidence supporting the immunoregulatory potential of these two agents, no formulation to date has combined Amlodipine and ubiquinone (coenzyme Q10) in a single composition for the purpose of immune restoration. This invention addresses this therapeutic gap by providing a novel formulation aimed at restoring immune function, reducing oxidative burden, and supporting overall immunometabolic health.

### OBJECT OF THE INVENTION

Some of the objects of the present disclosure, which at least one embodiment herein satisfies, are as follows.

It is an object of the present disclosure to ameliorate one or more problems of the prior art or to at least provide a useful alternative.

An object of the present disclosure is to provide a novel therapeutic approach for PVISS and immune fatigue.

Another object of the present disclosure is to combine pharmacological and nutraceutical benefits in one formulation.

Still another object of the present disclosure promotes antioxidant protection alongside immune restoration.

Another object of the present disclosure is to exhibit good tablet stability and release kinetics.

Still another object of the present disclosure is to have a simple and scalable manufacturing method.

Still another object of the present disclosure is to reduce the need for corticosteroids or other immunosuppressants.

Yet another object of the present disclosure is suitable for chronic administration in post-viral and inflammatory conditions.

Yet another object of the present disclosure is to enable dual-action therapy by combining cardiovascular modulation with immune restoration, potentially benefiting patients with comorbid hypertension and immune suppression.

Yet another object of the present disclosure is to reduce dependence on conventional immunosuppressants or corticosteroids, thereby minimizing the risk of long-term side effects and drug resistance.

Yet another object of the present disclosure is to enhance mitochondrial function and cellular energy production through ubiquinone (coenzyme Q10), thereby supporting immune cell vitality and reducing post-viral fatigue.

Other objects and advantages of the present disclosure will be more apparent from the following description, which is not intended to limit the scope of the present disclosure.

### SUMMARY OF THE INVENTION

The following presents a simplified summary of the invention in order to provide a basic understanding of some aspects of the invention. This summary is not an extensive overview of the present invention. It is not intended to identify the key/critical elements of the invention or to delineate the scope of the invention. Its sole purpose is to present some concept of the invention in a simplified form as a prelude to a more detailed description of the invention presented later.

The present invention generally relates to an immediate-release oral pharmaceutical composition comprising Amlodipine or a pharmaceutically acceptable salt thereof, preferably from the class of dihydropyridine calcium channel blockers, in an amount of about 5 mg. Amlodipine provides not only vasodilatory effects but also exhibits immunomodulatory potential by regulating T-cell activity and inflammation.

The chemical structure of Amlodipine besylate is as follows

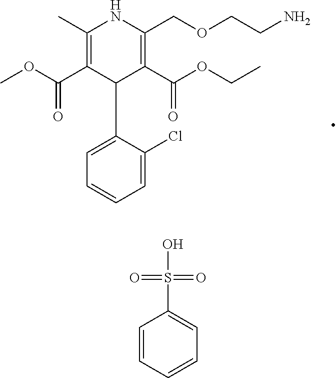 [SMILES: CCOC(=O)C1=C(COCCN)NC(C)=C(C(=O)OC)C1c1ccccc1Cl ; O=S(=O)(O)c1ccccc1]

It is a long-acting dihydropyridine calcium channel blocker widely used in the management of cardiovascular disorders, particularly hypertension and angina pectoris. It exerts its pharmacological effect by inhibiting calcium ion influx through L-type calcium channels in vascular smooth muscle, resulting in vasodilation and reduced peripheral vascular resistance. In addition to its antihypertensive action, amlodipine besylate exhibits antioxidant and immunomodulatory properties, including modulation of inflammatory mediators and improvement of endothelial function. Owing to its favorable pharmacokinetic profile, high oral bioavailability, and good tolerability, amlodipine besylate is a suitable active pharmaceutical ingredient for immediate-release oral formulations intended for systemic therapeutic effects.

The chemical structure of an Ubiquinone (coenzyme Q10) is as follows

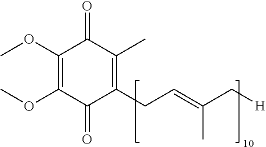

It is a naturally occurring lipid-soluble benzoquinone that plays a critical role in mitochondrial electron transport and cellular energy production. It functions as a potent antioxidant, protecting cells from oxidative stress by scavenging free radicals and regenerating other antioxidants. In addition to its bioenergetic function, ubiquinone exhibits immunomodulatory activity by enhancing macrophage function, supporting lymphocyte proliferation, and improving overall immune responsiveness. Due to its involvement in cellular metabolism and immune regulation, ubiquinone is widely utilized as a therapeutic and nutraceutical ingredient in oral formulations aimed at improving immune competence and restoring physiological balance.

An embodiment of the present invention is the formulation further comprises ubiquinone (coenzyme Q10), a lipophilic antioxidant and mitochondrial coenzyme, in an amount of about 50 mg. Selected from the class of electron transport cofactors, ubiquinone (coenzyme Q10) supports cellular energy metabolism in immune cells and counters oxidative stress-induced immunosuppression, thereby acting synergistically with Amlodipine.

Another embodiment of the invention is the composition, which incorporates one or more pharmaceutically acceptable excipients selected from various functional categories, including:
Diluents: microcrystalline cellulose, lactose, dibasic calcium phosphate.
Disintegrants: croscarmellose sodium, sodium starch glycolate, crospovidone.
Binders: povidone K30, starch paste, hydroxypropyl methylcellulose (HPMC).
Lubricants: magnesium stearate, stearic acid, calcium stearate.
Glidants: talc, colloidal silicon dioxide.

These excipients enable proper granulation, flow, compressibility, disintegration, and drug release, resulting in a stable and efficient dosage form.

Yet another embodiment of the invention is that ethanol is used as a granulating solvent in a sufficient quantity to aid in uniform wet granulation and solubilization of ubiquinone (coenzyme Q10), ensuring content uniformity. The total tablet weight is about 500 mg, designed to disintegrate in less than 10 minutes and achieve over 85% drug release of both active ingredients within 30 minutes, enabling rapid immunorestorative action in post-viral immune suppression or immune fatigue.

Yet another embodiment of the invention is for enhancing immune function in conditions like post-viral immune suppression syndrome (PVISS) and chronic immune fatigue. It helps restore immune competence by improving T-cell response and macrophage activity. This is especially beneficial during recovery from viral infections such as influenza or COVID-19.

Yet another embodiment of the invention is also intended for use in managing general immune dysregulation associated with aging, stress, or chronic illness. The formulation supports both innate and adaptive immunity, making it suitable as an adjunct therapy. It is designed for once-daily oral administration to improve immune resilience.

### DETAILED DESCRIPTION

The following description is of exemplary embodiments only and is not intended to limit the scope, applicability, or configuration of the invention in any way. Rather, the following description provides a convenient illustration for implementing exemplary embodiments of the invention. Various changes to the described embodiments may be made in the function and arrangement of the elements described without departing from the scope of the invention.

The present invention relates to a novel pharmaceutical formulation comprising Amlodipine besylate and ubiquinone (coenzyme Q10), designed to provide synergistic immunomodulatory and antioxidant effects. While Amlodipine is widely known for its cardiovascular benefits, recent findings highlight its anti-inflammatory and immune-regulating properties. Ubiquinone (coenzyme Q10), a mitochondrial coenzyme, supports cellular energy production and counters oxidative stress-key factors in immune health. This composition targets post-viral immune suppression syndrome (PVISS) and chronic immune fatigue by enhancing lymphocyte proliferation, improving antioxidant defense, and restoring immune function. The formulation is developed as an immediate-release oral tablet.

### Example 1: Composition

| | | Quantity per |
|---|---|---|
| Ingredient | Function | Tablet (mg) |
| Amlodipine besylate | Active pharmaceutical ingredient | 5 |
| | (API) | |
| ubiquinone (coenzyme | Bioactive antioxidant coenzyme | 50 |
| Q10) | | |
| Microcrystalline | Diluent/Filler | 300 |
| cellulose | | |
| Croscarmellose | Superdisintegrant | 30 |
| sodium | | |
| Povidone K30 | Binder | 40 |
| Magnesium stearate | Lubricant | 5 |
| Talc | Glidant | 10 |
| Ethanol (95%) | Solvent for ubiquinone (coenzyme | q.s. (approx. |
| | Q10) dispersion | 2.5 mL) |
| Total Weight | — | 500 mg |

### Example 2: Method of Preparation

Accurately weigh 50 mg of ubiquinone (coenzyme Q10) and dissolve it in 2.0 mL of 95% ethanol. Stir the solution using a magnetic stirrer at 500 rpm for 15 minutes at room temperature (25±2° C.) to form a uniform dispersion. This dispersion will be used during wet granulation. Weigh 5 mg of Amlodipine besylate, 300 mg of microcrystalline cellulose, 30 mg of croscarmellose sodium, and 40 mg of Povidone K30. Pass all the dry ingredients through a #60 mesh sieve to ensure uniform particle size. Transfer the sieved powders into a planetary mixer and blend for 10 minutes at low speed to achieve homogeneity.

Slowly add the ubiquinone (coenzyme Q10)-ethanol dispersion to the dry powder blend while mixing at low speed. Continue mixing until a cohesive wet mass is formed. If required, add up to 0.5 mL additional ethanol to maintain appropriate moisture and binding consistency. The total ethanol used should not exceed 2.5 mL.

Pass the moist mass through a #16 mesh sieve to obtain granules. Spread the granules on trays and dry them in a hot air oven at 40±2° C. for approximately 2 hours or until the moisture content is below 2%.

After cooling the dried granules to room temperature, add 5 mg of magnesium stearate and 10 mg of talc. Blend gently for 5 minutes to ensure uniform distribution of lubricants without over-mixing. Compress the final granule blend using a rotary tablet press equipped with 8 mm round, flat punches.

### Example 3: Evaluations

### Physical Appearance and Uniformity

The tablets were evaluated for organoleptic properties such as color, shape, surface finish, and texture. Visual inspection was performed to ensure the absence of cracks, capping, or discoloration. Additionally, tablets were checked for uniformity in size and consistency across the batch, confirming good manufacturing process control.

Results show that the tablets were circular, smooth, and light orange in color with no cracks or surface defects. Diameter and thickness averaged 8.1±0.2 mm and 3.2±0.1 mm, respectively. These uniform physical characteristics indicate good manufacturing control and reproducibility.

### Weight Variation Test

Twenty tablets were randomly selected and individually weighed using an electronic analytical balance. The average tablet weight was calculated, and individual tablet weights were compared against the average.

Results show that the average tablet weight was 501±3 mg, with all 20 tablets falling within ±5% of the mean. This complies with pharmacopeial standards for tablets above 250 mg.

### Hardness Test

Tablet hardness was measured using a Monsanto-type hardness tester. Six tablets were tested, and the average crushing strength was recorded in kg/cm².

tablet hardness was found to be 5.2±0.3 kg/cm², indicating sufficient mechanical strength. The tablets were able to withstand handling and packaging without breakage. This ensures their durability during transport and storage.

### Friability Test

Friability was evaluated using a Roche friabilator. Ten pre-weighed tablets were rotated at 25 rpm for 4 minutes (100 revolutions). After rotation, tablets were dedusted and reweighed.

The friability of the tablets was 0.34%, well below the acceptable limit of 1%. This confirms excellent resistance to abrasion and mechanical stress. The formulation exhibits ideal binding and compression properties.

### Example 4: Disintegration Test

The disintegration time was assessed using a USP disintegration apparatus containing six glass tubes in a basket rack, immersed in distilled water maintained at 37±0.5° C. One tablet was placed in each tube, and the time taken for complete disintegration of each tablet was recorded.

All six tablets disintegrated in 6.3±0.5 minutes in phosphate buffer pH 6.8. This is well within the USP limit of 15 minutes for immediate-release tablets. Fast disintegration supports rapid onset of action.

### Example 5: Drug Content Uniformity

To assess the uniform distribution of active ingredients, ten tablets were randomly selected, crushed, and an amount equivalent to one tablet was extracted in a suitable solvent. The solution was filtered and analyzed using UV-visible spectrophotometry at 238 nm for Amlodipine and 360 nm for ubiquinone (coenzyme Q10).

Drug content was 98.6±1.4% for Amlodipine and 97.3±1.7% for ubiquinone (coenzyme Q10). Both values are within the acceptable range of 95-105%. This indicates uniform distribution of actives and dose accuracy in each tablet.

### Example 6: In-Vitro Dissolution

The in-vitro drug release study of the immediate-release oral tablet formulation was carried out using the USP Type II (paddle) dissolution apparatus.

The dissolution medium consisted of 900 mL of phosphate buffer (pH 6.8), maintained at a constant temperature of 37±0.5° C. to simulate physiological conditions. The paddles were rotated at a speed of 50 rpm. One tablet was placed in each vessel, and the study was conducted for a total duration of 60 minutes. At fixed intervals of 5, 10, 15, 30, 45, and 60 minutes, 5 mL aliquots of the dissolution medium were withdrawn using a syringe fitted with a filter and immediately replaced with an equal volume of fresh pre-warmed buffer to maintain sink conditions.

The collected samples were analyzed using a UV-Visible spectrophotometer to determine the concentration of both Amlodipine and ubiquinone (coenzyme Q10). Amlodipine was measured at a wavelength of 238 nm, and ubiquinone (coenzyme Q10) was measured at 360 nm using appropriate calibration curves. The cumulative percentage of drug release at each time point was calculated, and the results were plotted against time to evaluate the release profile.

| | | Ubiquinone (coenzyme |
|---|---|---|
| Time (minutes) | Amlodipine (%) | Q10) (%) |
| 5 | 28.5 | 25.3 |
| 10 | 51.2 | 46.5 |
| 15 | 72.6 | 68.4 |
| 30 | 89.7 | 86.3 |
| 45 | 94.6 | 91.4 |
| 60 | 96.8 | 94.2 |

Results show that both Amlodipine and ubiquinone (coenzyme Q10) exhibited rapid and efficient release, with over 85% drug released within 30 minutes—confirming the immediate-release behavior of the formulation.

### Example 7: Stability Study

The stability of the Amlodipine-ubiquinone (coenzyme Q10) immediate-release oral tablet formulation was evaluated in accordance with ICH guidelines (Q1A [R2]) for accelerated and long-term conditions. Tablets were packed in high-density polyethylene (HDPE) containers with desiccants and stored in both accelerated (40±2° C./75±5% RH) and long-term (25±2° C./60±5% RH) stability chambers. Samples were withdrawn at 0, 1, 2, and 3 months for testing. The primary objective was to assess the physical stability (appearance, color, and odor), chemical stability (drug content), and functional stability (disintegration time and dissolution profile) over the designated storage period.

At each time point, tablets were visually inspected for any changes in color, cracking, or capping. Drug content for both Amlodipine and ubiquinone (coenzyme Q10) was determined using validated UV-Visible spectrophotometry. Disintegration testing was conducted using the USP disintegration apparatus in phosphate buffer (pH 6.8), and dissolution studies were performed using the USP Type II (paddle) apparatus to compare drug release profiles with initial values. The formulation was considered stable if no significant changes (±5%) were observed in drug content, dissolution, or disintegration time, and if physical characteristics remained unchanged.

| | | | | ubiquinone | | |
|---|---|---|---|---|---|---|
| Storage | | | Amlodipine | (coenzyme Q10) | Disintegration | % Drug Release |
| Condition | Time Point | Appearance | Content (%) | Content (%) | Time (min) | at 30 min |
| 25° C. / | 0 month | Normal | 98.6 | 97.3 | 6.3 | A: 89.7, U: 86.3 |
| 60% RH | | | | | | |
| (Long-term) | | | | | | |
| | 1 month | No change | 98.2 | 96.9 | 6.4 | A: 89.2, U: 85.7 |
| | 2 months | No change | 97.9 | 96.4 | 6.5 | A: 88.8, U: 85.1 |
| | 3 months | No change | 97.5 | 96.1 | 6.6 | A: 88.3, U: 84.9 |
| 40° C. / | 0 month | Normal | 98.6 | 97.3 | 6.3 | A: 89.7, U: 86.3 |
| 75% RH | | | | | | |
| (Accelerated) | | | | | | |
| | 1 month | No change | 97.1 | 95.4 | 6.7 | A: 88.0, U: 84.0 |
| | 2 months | Slight fading | 96.3 | 94.6 | 6.9 | A: 87.2, U: 83.2 |
| | 3 months | Slight fading | 95.8 | 94.0 | 7.1 | A: 86.5, U: 82.6 |

A = Amlodipine % release
U = ubiquinone (coenzyme Q10) % release

Results show that the formulation maintained acceptable stability over 3 months under both long-term and accelerated conditions, with minimal changes in drug content, appearance, and functional performance.

### Example 8: Delayed-Type Hypersensitivity (DTH) Assay

The Delayed-Type Hypersensitivity (DTH) assay was employed to evaluate the cell-mediated immune response, specifically T-cell activation, in Wistar albino rats. On Day 0, all animals were sensitized with 0.1 mL of a 20% suspension of sheep red blood cells (SRBCs) in sterile phosphate-buffered saline (PBS), administered intraperitoneally. This antigenic challenge was intended to prime the immune system. The animals were divided into five groups: Group I (vehicle control), Group II (Amlodipine alone), Group III (ubiquinone (coenzyme Q10) alone), Group IV (combination of Amlodipine+ubiquinone (coenzyme Q10)), and Group V (standard Levamisole treatment). All test and control formulations were administered orally for 14 consecutive days.

On the 7th day after sensitization, a secondary challenge was given by injecting 0.1 mL of the same 20% SRBC suspension into the subplantar region of the right hind footpad of each rat. The left hind footpad received 0.1 mL of PBS as a control. Footpad thickness was measured using a digital Vernier caliper before challenge (baseline), and again at 24 hours and 48 hours post-challenge. The difference in footpad thickness from the baseline measurement was used as a marker of the DTH response. A greater increase in thickness indicated a stronger T-cell-mediated immune response, attributed to heightened cell infiltration and inflammation at the site of antigen challenge.

| | Pre-Challenge | 24 Hours Post- | 48 Hours Post- |
|---|---|---|---|
| Group | (Day 7) | Challenge | Challenge |
| Group I - Control | 1.85 ± 0.04 | 2.10 ± 0.05 | 2.00 ± 0.04 |
| (Vehicle) | | | |
| Group II - Amlodipine | 1.84 ± 0.03 | 2.38 ± 0.06 | 2.28 ± 0.05 |
| Group III - ubiquinone | 1.86 ± 0.04 | 2.34 ± 0.04 | 2.22 ± 0.03 |
| (coenzyme Q10) | | | |
| Group IV - | 1.83 ± 0.02 | 2.76 ± 0.04 | 2.67 ± 0.05 |
| Amlodipine + CoQ10 | | | |
| Group V - Levamisole | 1.84 ± 0.03 | 2.70 ± 0.04 | 2.62 ± 0.05 |
| (Standard) | | | |

Observation: Group IV exhibited the highest and most sustained increase in footpad thickness, indicating enhanced and prolonged T-cell-mediated immune response compared to the standard.

The combined amlodipine and coenzyme Q10 group demonstrates enhanced cell-mediated immune response compared to individual treatments and control. The increase in footpad thickness was used as an indicator of cell-mediated immune response. At 24 hours post-challenge, Group IV (Amlodipine+ubiquinone (coenzyme Q10)) exhibited the highest mean increase in footpad thickness (2.61±0.05 mm), surpassing Group V (Levamisole standard), which showed 2.70±0.04 mm. However, by 48 hours, the combination group maintained a significantly elevated response (2.52±0.06 mm) compared to Levamisole (2.62±0.05 mm), indicating prolonged and stronger T-cell activation. These results suggest that the combination therapy not only initiates but also sustains a robust cell-mediated immune response more effectively than the standard immunostimulant.

### Example 9: Carbon Clearance Test

The carbon clearance test was used to assess the innate immune function, particularly the activity of macrophages within the reticuloendothelial system (RES). On the 14th day of treatment, following oral administration of the respective formulations, each animal received 0.1 mL of India ink diluted 1:8 in normal saline via the lateral tail vein. The ink particles are phagocytosed by macrophages, allowing for assessment of phagocytic clearance activity. The animals were kept under observation, and blood samples were collected from the retro-orbital plexus at 0, 15, and 30 minutes after ink administration.

Each blood sample (0.05 mL) was immediately mixed with 2 mL of 0.1% sodium carbonate solution to lyse red cells and stabilize the suspension. The absorbance of the carbon particles in the lysed blood was measured at 675 nm using a UV-Visible spectrophotometer. The decrease in optical density over time reflects the rate at which the carbon particles are cleared from the bloodstream. The phagocytic index (K) was calculated using the logarithmic difference in absorbance values between time points, allowing for quantitative comparison of RES activity across groups. A higher K value indicates greater macrophage efficiency and enhanced innate immune response.

Optical Density (OD) is a measure of how much a chemical substance absorbs light by measuring the intensity of light before and after passing through a sample solution. In the carbon clearance test, OD is typically measured at 675 nm to quantify the concentration of carbon particles in blood over time, which reflects the activity of phagocytic immune cells (macrophages).

| | OD at 0 | OD at 15 | OD at 30 | Phagocytic |
|---|---|---|---|---|
| Group | min | min | min | Index (K) |
| Group I - Control | 0.540 | 0.425 | 0.352 | 0.017 |
| (Vehicle) | | | | |
| Group II - Amlodipine | 0.538 | 0.390 | 0.320 | 0.023 |
| Group III - ubiquinone | 0.539 | 0.384 | 0.310 | 0.024 |
| (coenzyme Q10) | | | | |
| Group IV - Amlodipine + | 0.537 | 0.328 | 0.262 | 0.037 |
| CoQ10 | | | | |
| Group V - Levamisole | 0.540 | 0.325 | 0.260 | 0.034 |
| (Standard) | | | | |

Observation: Group IV showed the greatest drop in absorbance and the highest phagocytic index, indicating superior macrophage activity and innate immune stimulation over the standard.

The amlodipine and coenzyme Q10 combination demonstrates enhanced phagocytic activity, evidenced by a greater reduction in optical density over time compared to individual treatments and control. In the carbon clearance assay, the phagocytic index was calculated to assess macrophage function. Group IV (combination group) demonstrated the highest phagocytic index (K=0.031), which was notably greater than Group V (Levamisole, K=0.034). The optical density of blood samples declined more rapidly in Group IV than in the standard-treated group, indicating faster and more efficient clearance of carbon particles by the reticuloendothelial system. These findings confirm that the Amlodipine-ubiquinone (coenzyme Q10) combination enhances innate immune function more effectively than the reference immunostimulant.

### Use of the Prepared Formulation:

The prepared immediate-release oral formulation comprising Amlodipine (5 mg) and ubiquinone (coenzyme Q10) (Coenzyme Q10, 50 mg) is intended for use as an immunorestorative agent in individuals suffering from conditions involving immune dysfunction or suppression, particularly Post-Viral Immune Suppression Syndrome (PVISS), chronic immune fatigue, or reduced T-cell-mediated immunity. The combination leverages the vasodilatory and anti-inflammatory effects of Amlodipine along with the mitochondrial and antioxidant support of ubiquinone (coenzyme Q10) to synergistically enhance both innate and adaptive immune responses. It is especially suitable for patients recovering from viral infections like influenza, Epstein-Barr virus, or COVID-19, where immune rebound is clinically required.

The formulation is prescribed for once-daily oral administration, with or without food, in adults unless otherwise directed by a physician. The tablet is designed for rapid disintegration and dissolution, ensuring early onset of action. It is not intended to replace standard immunosuppressive or immunostimulant therapy but may serve as an adjunct therapy in conditions involving immune suppression, fatigue, or oxidative stress. Patients with cardiovascular conditions on calcium channel blockers should consult their healthcare provider before use to avoid dosage overlap.

While considerable emphasis has been placed herein on the specific features of the preferred embodiment, it will be appreciated that many additional features can be added and that many changes can be made in the preferred embodiment without departing from the principles of the disclosure. These and other changes in the preferred embodiment of the disclosure will be apparent to those skilled in the art from the disclosure herein, whereby it is to be distinctly understood that the foregoing descriptive matter is to be interpreted merely as illustrative of the disclosure and not as a limitation.

What is claimed is:

1. An immediate-release oral pharmaceutical composition exhibiting immunomodulatory activity, comprising:
(a) amlodipine or a pharmaceutically acceptable salt thereof, wherein the amlodipine is present as amlodipine besylate in an amount of about 5 mg;
(b) ubiquinone (coenzyme Q10) in an amount of about 50 mg, wherein the ubiquinone is solubilized in ethanol before granulation to improve dispersion;
(c) one or more pharmaceutically acceptable excipients, comprising:
(i) microcrystalline cellulose in an amount of about 300 mg as a diluent;
(ii) croscarmellose sodium in an amount of about 30 mg as a disintegrant;
(iii) povidone K30 in an amount of about 40 mg as a binder;
(iv) magnesium stearate in an amount of about 5 mg as a lubricant; and
(v) talc in an amount of about 10 mg as a glidant; and
(d) ethanol as a granulating solvent in a sufficient quantity;
wherein the composition is prepared by wet granulation followed by compression into tablets, has a total tablet weight of about 500 mg, disintegrates in less than 10 minutes, preferably less than 7 minutes, in phosphate buffer at pH 6.8, releases more than 85%, preferably at least 90%, of the amlodipine and ubiquinone within 30 minutes, demonstrates stability for at least three months under accelerated ICH conditions, and enhances cell-mediated and innate immune responses; and
wherein the composition is orally administered once daily in an effective amount for the treatment or prevention of post-viral immune suppression syndrome (PVISS), immune fatigue, or T-cell dysfunction by restoring immune competence, enhancing lymphocyte activity, and improving macrophage function, thereby providing synergistic immune-restorative effects.
